(19) United States
(12) Patent Application Publication
(10) Pub. No.: US 2007/0297997 A1
(43) Pub. Date: Dec. 27, 2007

(54) Personal care composition

(75) Inventor: Paul Robert Tanner, Lebanon, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(73) Assignee: The Procter & Gamble Company

(21) Appl. No.: 11/472,739

(22) Filed: Jun. 22, 2006

(51) Int. Cl. A61K 8/89; A61K 8/37

(52) U.S. Cl. 424/59; 424/70.12

(57) ABSTRACT

Stable personal care composition comprising from about 1% to about 20% of a non-emulsifying silicone elastomer; from about 0.5% to about 10% of an emulsifying silicone elastomer; from about 5% to about 30% of an oil-soluble sunscreen; from about 1% to about 20% of a sunscreen solvent; and from about 30% to about 80% of a non-polar emollient.

### FIELD OF THE INVENTION

The present invention relates to a non-emulsified personal care composition comprising an organic sunscreen and silicone elastomers.

### BACKGROUND OF THE INVENTION

An ongoing need exists to provide personal care compositions which prevent damage to the skin and other keratinous tissue from harmful ultraviolet radiation. To provide effective protection, such compositions must be regularly applied. An additional ongoing need exists, therefore, to provide sunscreen compositions that have a pleasant feel, and thus encourage frequent use.

Oily sunscreens, including oil-soluble crystalline sunscreens, are desirable for their effectiveness, yet have an unpleasant skin feel and other undesirable characteristics associated with applying an oily composition to the skin. Thickening oily sunscreens remains challenging, in particular when the sunscreen is present at higher, more effective concentrations. Whereas it may be possible to thicken an oil phase by using various types of waxy materials, these tend to exhibit instability at higher temperatures. Sunscreen compositions in the form of an emulsion may have a less oily feel. However, emulsions comprise water, emulsifiers, and aqueous thickeners. Water may promote the growth of microbes and thus shorten the shelf-life of the composition. Preservatives, emulsifiers and aqueous thickeners may have an unpleasant skin feel and/or may be irritating to the skin.

There exists a need, therefore, to provide sunscreen compositions which effectively protect against ultraviolet radiation, which have a pleasant feel and other characteristics so as to encourage frequent use, and which are not irritating to the skin.

### SUMMARY OF THE INVENTION

The present invention meets the aforementioned needs by providing a non-emulsified skin care composition comprising an oil-soluble sunscreen, a non-polar emollient, a suitable sunscreen solvent, and emulsifying and non-emulsifying silicone elastomers. The composition of the present invention provides a number of advantages. Because the composition is non-emulsified, the need for potentially irritating and unpleasant-feeling emulsifiers and preservatives is eliminated. The sunscreen solvent solubilizes oily and crystalline sunscreens, and also is compatible with the silicone elastomers, which thicken the composition while providing a pleasant skin feel. The pleasant feel encourages frequent use of a sunscreen active, which provides a chronic benefit in the form of protection from harmful UV-radiation. In addition, unlike compositions which comprise waxy materials as primary thickeners, the composition of the present invention is stable at higher temperatures and may not require heating during the manufacturing process (i.e. a “cold-filling” process may be used). This results in a lower cost and increased efficiency of production.

The following represent some non-limiting embodiments of the present invention.

According to the first embodiment of the present invention, a personal care composition is provided comprising from about 1% to about 20% of a non-emulsifying silicone elastomer; from about 0.5% to about 10% of an emulsifying silicone elastomer; from about 5% to about 30% of an oil-soluble sunscreen; from about 30% to about 80% of a non-polar emollient, and from about 1% to about 20% of a sunscreen solvent. In one embodiment, the composition is substantially anhydrous.

According to another embodiment of the present invention, a method of providing a benefit to mammalian keratinous tissue in need thereof is provided, comprising the step of applying the composition of the first embodiment to keratinous tissue.

According to another embodiment of the present invention, a kit is provided, comprising the composition of the first embodiment.

### DETAILED DESCRIPTION OF THE INVENTION

The composition of the present invention may be used in skin care, cosmetic, and hair care products, non-limiting uses of which include moisturizers, conditioners, anti-aging compounds, skin lightening compounds, and combinations thereof. In one embodiment, the composition is applied to the face, neck, hands, arms and other areas of the body exposed to ultraviolet radiation. Alternatively, the composition is applied to insult-affected areas of keratinous tissue.

In all embodiments of the present invention, all percentages are by weight of the composition, unless otherwise specified. All ratios are weight ratios, unless specifically stated otherwise. The number of significant digits conveys neither limitations on the indicated amounts nor on the accuracy of the measurements. All amounts indicating quantities, percentages, proportions, etc. are understood to be modified by the word “about” unless otherwise specifically indicated. All measurements are understood to be made at about 25° C. and at ambient conditions, where “ambient conditions” means conditions under about one atmosphere of pressure and at about 50% relative humidity.

Herein, “personal care composition” means compositions suitable for topical application on mammalian keratinous tissue. The personal care compositions described herein may contain one or more skin care actives. “Skin care actives,” or “actives,” as used herein, means compounds that aid in regulating the condition of skin and of other mammalian keratinous tissue, for example, by providing a benefit or improvement to the keratinous tissue.

“Keratinous tissue,” as used herein, refers to keratin-containing layers disposed as the outermost protective covering of mammals which includes, but is not limited to, skin, hair, nails, cuticles, etc.

Herein, “stable” and “stability” mean compositions which are substantially unaltered in chemical state, physical homogeneity and/or color, upon exposure to conditions reasonably expected to be incurred in shipping, storage and use, for example, for at least 30 days at a temperature of from about 0° C. to about 40° C.

“Substantially free,” as used herein, unless otherwise specified means that the compound of which the composition is substantially free is not added to the composition or phase; however, small amounts incidentally may be present, for example as a by-product of chemical reactions, or as a result of absorption from the surroundings. Alternatively, substantially free may be understood to mean less than about 1%, by weight of the phase.

“Substantially anhydrous,” as used herein, means that water is not added to the composition; however, small amounts (typically less than 1%) incidentally may be present, for example, as a byproduct of chemical reactions, or as a result of absorption from the surroundings.

“Derivatives,” as used herein, means ester, ether, amide and/or salt derivatives of the relevant compound.

Herein, “regulating the condition of keratinous tissue” means improving the condition of mammalian keratinous tissue and/or prophylactically regulating the condition of mammalian keratinous tissue, and includes, for example, protecting the tissue from ultraviolet radiation, and regulating the signs of skin aging. Herein, “improving the condition of mammalian keratinous tissue” means effecting a visually and/or tactilely perceptible positive change in the appearance and feel of the tissue. Conditions that may be regulated and/or improved include, but are not limited to, one or more of the following: increasing the luminosity, or “glow” of the skin, reducing the appearance of wrinkles and coarse deep lines, fine lines, crevices, bumps, and large pores; thickening of keratinous tissue (e.g., building the epidermis and/or dermis and/or sub-dermal layers of the skin, and where applicable the keratinous layers of the nail and hair shaft, to reduce skin, hair, or nail atrophy); increasing the convolution of the dermal-epidermal border (also known as the rete ridges); preventing loss of skin or hair elasticity, for example, due to loss, damage and/or inactivation of functional skin elastin, resulting in such conditions as elastosis, sagging, loss of skin or hair recoil from deformation; reduction in cellulite; change in coloration to the skin, hair, or nails, for example, under-eye circles, blotchiness (e.g., uneven red coloration due to, for example, rosacea), sallowness, discoloration caused by telangiectasia or spider vessels, dryness, brittleness, and graying hair.

As used herein, “signs of skin aging,” include, but are not limited to, outward visibly and tactilely perceptible manifestations, as well as any macro- or microeffects, due to keratinous tissue aging. These signs may result from processes which include, but are not limited to, the development of textural discontinuities such as wrinkles and coarse deep wrinkles, fine lines, skin lines, crevices, bumps, large pores, unevenness or roughness; flaking; dryness; loss of skin elasticity; discoloration (including undereye circles); blotchiness; dullness or lack of luminosity, sallowness; hyperpigmented skin regions such as age spots and freckles; keratoses; abnormal differentiation; hyperkeratinization; elastosis; collagen breakdown, and other histological changes in the stratum corneum, dermis, epidermis, vascular system (e.g., telangiectasia or spider vessels), and underlying tissues (e.g., fat and/or muscle), especially those proximate to the skin.

“Dermatologically-acceptable,” as used herein, means that the compositions or components thereof so described are suitable for use in contact with mammalian keratinous tissue without undue toxicity, incompatibility, instability, allergic response, and the like.

“Insult-affected keratinous tissue,” as used herein, means keratinous tissue which exhibits discomfort, irritation, an unpleasant or irregular appearance and the like, for example after exposure to a physical and/or chemical irritant. In one embodiment, the insult-affected keratinous tissue is human skin. Non-limiting examples of insult-affected keratinous tissue include sunburn and other types of burns; rashes, such as diaper rash, shaving rash and allergen-induced rashes; discoloration, such as bleaching, staining or hyperpigmentation; nicks and cuts due to, for example, shaving; dry, chapped or rough skin due to exposure to example wind, cold and/or low humidity, etc. Non-limiting examples of insults include ultraviolet radiation, wind, low humidity, allergens, pollutants, chemical and natural irritants, bodily fluids, bodily waste, excessive moisture, bacteria, fungi, etc.

Herein “kit” means a packaging unit comprising at least one composition described herein. The kit may comprise a plurality of components, including a composition described herein. The kit further may comprise one or more additional compositions, suitable for application prior to or after application of the composition described herein, one or more orally ingestible dietary supplements, a delivery enhancement device, instructions for use of the device, instructions for complying with suitable application regimens, and combinations thereof.

### I. Composition

The composition of the present invention may have a viscosity of from about 10,000 cps (centipoise) to about 1,000,000 cps, alternatively from about 30,000 cps to about 500,000 cps, and alternatively from about 40,000 cps to about 200,000 cps.

The composition may be substantially free from surfactants and/or emulsifiers. Additionally or alternatively, the composition may be substantially free from waxes. When the composition is substantially free from waxes, and alternatively comprises less than about 1% by weight of the phase of one or more waxes, the composition may exhibit stability at a temperature of up to about 45° C., and the manufacturing process may proceed occur without the step of heating the composition above room temperature, or about 25° C. (also referred to as “cold processing.”)

In one embodiment, the composition is substantially anhydrous, to increase stability of the sunscreen and of the composition, and to increase the pleasant feel characteristics of the composition.

### A. Silicone Elastomer

The composition of the present invention comprises a silicone elastomer, useful for reducing the tackiness of the composition and for providing a pleasant feel upon application. One non-limiting example of useful silicone elastomers is crosslinked organopolysiloxane (or siloxane) elastomers, as described in U.S. patent publication 2003/0049212A1. The elastomers may comprise emulsifying and non-emulsifying silicone elastomers. “Emulsifying,” as used herein, means crosslinked organopolysiloxane elastomers having at least one polyoxyalkylene (e.g., polyoxyethylene or polyoxypropylene) or polyglycerin moiety, whereas “non-emulsifying” means crosslinked organopolysiloxane elastomers essentially free of polyoxyalkylene or polyglycerin moeities.

The composition of the present invention may comprise from about 1% to about 20%, and alternatively from about 5% to about 15%, of a non-emulsifying crosslinked organopolysiloxane elastomer. Non-limiting examples of suitable non-emulsifying crosslinked organopolysiloxane elastomers include dimethicone crosspolymers, dimethicone/vinyl dimethicone crosspolymers, and copolymers, derivatives and mixtures thereof, supplied by Dow Corning™ (e.g. DC 9040, 9041, 9045, 8509, 9546, 9506); C30-45 alkyl cetearyl dimethicone crosspolymer, cetearyl dimethicone crosspolymer, and copolymers, derivatives and mixtures thereof, supplied by General Electric™ (e.g. Velvesil™ 125 and Velvesil™ DM); dimethicone/phenyl vinyl dimethicone crosspolymer, vinyl dimethicone/lauryl dimethicone crosspolymer, trifluoropropyl dimethicone/trifluoropropyl divinyldimethicone crosspolymer, and copolymers, derivatives and mixtures thereof, supplied by Shin EtSu™ (KSG-15, -15AP, -16, -17, -18, -41, -42, -43, -44, -51, -103), and the Grant Industries line of elastomers, available as GRANSIL™.

The composition of the present invention may comprise from about 0.5% to about 10%, and alternatively from about 1% to about 5%, of an emulsifying crosslinked organopolysiloxane elastomer, described in U.S. Pat. Nos. 5,412,004; 5,837,793; and 5,811,487. Non-limiting examples of suitable emulsifying elastomers include PEG-12 dimethicone crosspolymers, supplied by Dow Corning™ (DC 9010 and 9011) dimethicone/PEG-10/15 crosspolymer, dimethicone/PEG-10 crosspolymer, PEG-15/lauryl dimethicone crosspolymer, trifluoropropyl dimethicone/PEG-10 crosspolymer, dimethicone/polyglycerin-3 crosspolymer, lauryl dimethicone/polyglycerin-3 crosspolymer, all supplied by Shin EtSu™ (KSG-24, -61, -21/210, -31/310, -32/320, -33/330, -34/340, -710, -810, -820, -830, and -840); polyoxyalkylene-modified elastomers formed from divinyl compounds, e.g. siloxane polymers with at least two free vinyl groups bonded via Si—H linkages on a polysiloxane backbone. In one embodiment, the emulsifying crosslinked organopolysiloxane elastomers are dimethyl polysiloxanes crosslinked by Si—H sites on a molecularly spherical MQ resin (R3SiO_{1/2} SiO_{4/2}), and alternatively is dimethicone copolyol crosspolymer and dimethicone, commercially available from Shin Etsu as KSG-21.

### B. Oil-Soluble Sunscreen

The composition of the present invention comprises from about 5% to about 30%, alternatively from about 5% to about 20%, and alternatively from about 5% to about 15%, of at least one oil-soluble sunscreen. Herein, “sunscreen” is understood to include both sunscreens and UV-light absorbers. In one embodiment, the composition comprises at least one sunscreen which, in its commercially-available purified form, is an oil-soluble crystalline and/or solid compound. It is to be understood that the crystalline sunscreen is substantially dissolved, and thus does not remain in a crystalline form, in the final composition. In one embodiment, the composition comprises at least 2%, alternatively from about 2% to about 15%, and alternatively from about 2% to about 10%, of an oil-soluble, crystalline sunscreen. Non-limiting examples of suitable oil-soluble sunscreens are disclosed in The Cosmetic, Toiletry, and Fragrance Association's The International Cosmetic Ingredient Dictionary and Handbook, 10^{th} Ed., Gottschalck, T. E. and McEwen, Jr., Eds. (2004), p. 2267 and pp. 2292-93 and include benzophenone-3, bis-ethylhexyloxyphenol methoxyphenyl triazine, butyl methoxydibenzoyl-methane, diethylamino hydroxy-benzoyl hexyl benzoate, drometrizole trisiloxane, ethylhexyl methoxy-cinnamate, ethylhexyl salicylate, ethylhexyl triazone, octocrylene, homosalate, polysilicone-15, oxybenzone, and derivatives and mixtures thereof.

The composition further may comprise from about 0.001% to about 10%, and alternatively from about 0.1% to about 5%, of an inorganic and/or oil-insoluble sunscreen. Non-limiting examples of suitable insoluble sunscreens include methylene bis-benzotriazolyl tetramethylbutyl-phenol, titanium dioxides, zinc cerium oxides, zinc oxides, and derivatives and mixtures thereof. It is to be understood that “oil-insoluble sunscreen” does not encompass water-soluble sunscreens.

### C. Non-Polar Emollient

The composition of the present invention comprises from about 30% to about 80% of a non-polar emollient. Non-limiting examples of suitable non-polar emollients include silicone oils, hydrocarbon oils, and mixtures thereof. Useful non-polar emollients in the present invention include natural, synthetic, saturated, unsaturated, straight chained, branched chained, linear, cyclic, aromatic, volatile, and non-volatile non-polar emollients, and mixtures thereof. “Non-polar,” as used herein, means that the solvent has an average solubility parameter of from about 5 to about 8 (cal/cm³)^{0.5}, where “cal” means calories. Solubility parameters are discussed in more detail by C. D. Vaughan in “The Solubility Parameter: What is it?,” Cosmetics & Toiletries vol. 106, November, 1991, pp. 69-72.

Non-limiting examples of suitable non-polar hydrocarbons include mineral oils and branched chain hydrocarbons. Mineral oils include petroleum derivatives which are mixtures of paraffinic and naphthenic (cyclic) hydrocarbons. These include both “light” and “heavy” mineral oils, which are differentiated on the basis of the average molecular weight of the hydrocarbon mixture.

Non-limiting examples of suitable branched chain hydrocarbons include hydrocarbon liquids containing from about 10 to about 68 carbons, and alternatively from 10 to about 24 carbons, commercially available, for example, under the tradenames Permethyl™ (Permethyl Corporation™) and Isopar™ (Exxon™).

Non-limiting examples of suitable non-polar silicones useful in the present invention include those disclosed in U.S. Pat. No. 4,781,917 issued to Luebbe et al. Additionally, a description of various silicones materials is found in Todd et al., “Volatile Silicone Fluids for Cosmetics”, Cosmetics and Toiletries, 91:27-32 (1976). In one embodiment, the silicone oils are selected from the group consisting of cyclic silicones corresponding to the formula:

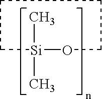

wherein n is from about 3 to about 7; and linear silicones corresponding to the formula:

(CH₃)₃ Si—O—[Si(CH₃)₂—O]ₘ—Si(CH₃)₃

wherein m is from about 0 to about 1000. Commercially available examples of these types of silicones include the Dow Corning 200 series, Dow Corning 344, and Dow Corning 345 (all available from Dow Corning™ Corp.); and SF1202, SF1204, and the Viscasil™ series (all available from the G.E. Silicones™).

Non-limiting examples of additional non-polar silicones useful in the present invention include alkyl (for example, 2 carbons to 30 carbons) and aryl (for example, phenyl or styrenyl) substituted silicones, including by not limited to phenyl methicone, phenyl dimethicone, phenyl trimethicone, diphenyl dimethicone, phenylethyl dimethicone, hexyl dimethicone, lauryl dimethicone, cetyl dimethicone, stearyl dimethicone, bis-stearyl dimethicone, and mixtures thereof.

### D. Sunscreen Solvent

The composition of the present invention comprises from about 1% to about 20%, and alternatively from about 5% to about 15%, of a sunscreen solvent, which is compatible with the non-polar emollients and elastomers described herein, and is suitable to substantially solubilize the oil-soluble sunscreen. “Sunscreen solvent” as used herein, may be understood to mean that in a solution comprising about 90% of the neat sunscreen solvent and about 10% of butyl methoxydibenzoyl-methane, both by weight of the composition, the butyl methoxydibenzoyl-methane is substantially soluble at room temperature, i.e. is no longer visible as a solid material to the naked eye by one having unimpaired vision.

Non-limiting examples of suitable sunscreen solvents include butyl and isopropyl phthalimide (Pelemol™ BIP), phenylethyl benzoate (X-tend™ 226), dicaprylyl carbonate (Tegosof™ DEC), isopropyl lauroylsarcosinate (Eldew™ SL 205), butyl octylsalicylate (Hallbrite™ BHB), dioctyl malate, dicaprylyl maleate (Hallbrite™ DCM), and derivatives and mixtures thereof.

### E. Optional Ingredients

### 1. Particulate Material

The compositions of the present invention may comprise from about 0.001% to about 40%, alternatively from about 3% to about 30%, and alternatively from about 5% to about 20%, of one or more particulate materials and/or cosmetic powders. Non-limiting examples of suitable powders include inorganic powders (for example, iron oxides, titanium dioxides, zinc oxides, silica), organic powders, composite powders, optical brightener particles, and mixtures of any of the foregoing. These particulates can, for instance, be platelet shaped, spherical, elongated or needle-shaped, or irregularly shaped; surface coated or uncoated; porous or non-porous; charged or uncharged; and can be added to the current compositions as a powder or as a pre-dispersion. In one embodiment, the particulate material is hydrophobically coated.

Suitable organic powders particulate materials include, but are not limited, to polymeric particles chosen from the methylsilsesquioxane resin microspheres, for example, Tospearl™ 145A, (Toshiba Silicone); microspheres of polymethylmethacrylates, for example, Micropearl™ M 100 (Seppic); the spherical particles of crosslinked polydimethylsiloxanes, for example, Trefil™ E 506C or Trefil™ E 505C (Dow Corning Toray Silicone); sphericle particles of polyamide, for example, nylon-12, and Orgasol™ 2002D Nat C05 (Atochem); polystyrene microspheres, for example Dyno Particles, sold under the name Dynospheres™, and ethylene acrylate copolymer, sold under the name FloBead™ EA209 (Kobo); aluminium starch octenylsuccinate, for example Dry Flo™ (National Starch); microspheres of polyethylene, for example Microthene™ FN510-00 (Equistar), silicone resin, polymethylsilsesquioxane silicone polymer, platelet shaped powder made from L-lauroyl lysine, and mixtures thereof.

The composition of the present invention further may comprise interference pigments, including hydrophobically-modified interference pigments. Herein, “interference pigments” means thin, platelike layered particles having two or more layers of controlled thickness. The layers have different refractive indices that yield a characteristic reflected color from the interference of typically two, but occasionally more, light reflections, from different layers of the platelike particle. One example of interference pigments are micas layered with about 50-300 nm films of TiO₂, Fe₂O₃, silica, tin oxide, and/or Cr₂O₃ and include pearlescent pigments. Intereference pigments are available commercially from a wide variety of suppliers, for example, Rona (Timiron™ and Dichrona™), Presperse (Flonac™), Englehard (Duochrome™), Kobo (SK-45-R and SK-45-G), BASF (Sicopearls™) and Eckart (Prestige™). In one embodiment, the average diameter of the longest side of the individual particles of interference pigments is less than about 75 microns, and alternatively less than about 50 microns.

### 2. Colorants

The composition of the present invention may comprise from about 0.001% to about 25%, and alternatively from about 0.01% to about 5%, of a colorant. The colorant may be oil-soluble, or may be hydrophobically modified to increase oil-solubility. Non-limiting classes of suitable colorants include, but are not limited to organic and/or inorganic pigments, natural and/or synthetic dyes, lakes, including FD&C and/or D&C lakes and blends, and mixtures of any of the foregoing.

Non-limiting examples of suitable colorants include iron oxides, ferric ammonium ferrocyanide, manganese violet, ultramarine blue, and chromium oxide, phthalocyanine blue and green pigment, encapsulated dyes, inorganic white pigments, for example TiO₂, ZnO, or ZrO₂, FD&C dyes, D&C dyes, and mixtures thereof.

### 4. Additional Skin Care Active

The composition of the present invention may comprise at least one additional skin care active. The skin care active should be soluble in and compatible with the elastomers and other components of the composition to produce a dermatologically-acceptable composition. Solubility is within the knowledge of one of skill in the art, and can be determined using known methods of analysis. One of skill in the art further will understand that solubility may be affected by the concentration of the skin care active, and the type and concentration of other components in the composition. Many skin care actives may provide more than one benefit, or operate via more than one mode of action; therefore, classifications herein are made for the sake of convenience and are not intended to limit the active to that particular application or applications listed.

### Vitamins

The compositions of the present invention may comprise from about 0.0001% to about 50%, alternatively from about 0.001% to about 10%, and alternatively from about 0.01% to about 5%, of one or more vitamins. Herein, “vitamins” means vitamins, pro-vitamins, and their salts, isomers and derivatives. Non-limiting examples of suitable vitamins include: vitamin B compounds (including B1 compounds, B2 compounds, B3 compounds such as niacinamide, niacinnicotinic acid, tocopheryl nicotinate, C₁-C₁₈ nicotinic acid esters, and nicotinyl alcohol; B5 compounds, such as panthenol or “pro-B5”, pantothenic acid, pantothenyl; B6 compounds, such as pyroxidine, pyridoxal, pyridoxamine; camitine, thiamine, riboflavin); vitamin A compounds, and all natural and/or synthetic analogs of Vitamin A, including retinoids, retinol, retinyl acetate, retinyl palmitate, retinoic acid, retinaldehyde, retinyl propionate, carotenoids (pro-vitamin A), and other compounds which possess the biological activity of Vitamin A; vitamin D compounds; vitamin K compounds; vitamin E compounds, or tocopherol, including tocopherol sorbate, tocopherol acetate, other esters of tocopherol and tocopheryl compounds; vitamin C compounds, including ascorbate, ascorbyl esters of fatty acids, and ascorbic acid derivatives, for example, ascorbyl phosphates such as magnesium ascorbyl phosphate and sodium ascorbyl phosphate, ascorbyl glucoside, and ascorbyl sorbate; and vitamin F compounds, such as saturated and/or unsaturated fatty acids. In one embodiment, the composition comprises a vitamin selected from the group consisting of vitamin B compounds, vitamin C compounds, vitamin E compounds and mixtures thereof. Alternatively, the vitamin is selected from the group consisting of niacinamide, tocopheryl nicotinate, pyroxidine, panthenol, vitamin E, vitamin E acetate, ascorbyl phosphates, ascorbyl glucoside, and mixtures thereof.

### Peptides and Peptide Derivatives

The compositions of the present invention may comprise one or more peptides. Herein, “peptide” refers to peptides containing ten or fewer amino acids, their derivatives, isomers, and complexes with other species such as metal ions (for example, copper, zinc, manganese, and magnesium). As used herein, peptide refers to both naturally occurring and synthesized peptides. In one embodiment, the peptides are di-, tri-, tetra-, penta-, and hexa-peptides, their salts, isomers, derivatives, and mixtures thereof. Examples of useful peptide derivatives include, but are not limited to, peptides derived from soy proteins, carnosine (beta-alanine-histidine), palmitoyl-lysine-threonine (pal-KT) and palmitoyl-lysine-threonine-threonine-lysine-serine (pal-KTTKS, available in a composition known as MATRIXYL®), palmitoyl-glycine-glutamine-proline-arginine (pal-GQPR, available in a composition known as RIGIN®), these three being available from Sederma, France, acetyl-glutamate-glutamate-methionine-glutamine-arginine-arginine (Ac-EEMQRR; Argireline®), and Cu-histidine-glycine-glycine (Cu-HGG, also known as IAMIN®).

The compositions may comprise from about 1×10⁻⁷% to about 20%, alternatively from about 1×10⁻⁶% to about 10%, and alternatively from about 1×10⁻⁵% to about 5% of the peptide.

### Sugar Amines

The compositions of the present invention may comprise a sugar amine, also known as amino sugars, and their salts, isomers, tautomers and derivatives. Sugar amines can be synthetic or natural in origin and can be used as pure compounds or as mixtures of compounds (e.g., extracts from natural sources or mixtures of synthetic materials). For example, glucosamine is generally found in many shellfish and can also be derived from fungal sources. Sugar amine compounds useful in the present invention include, for example, N-acetyl-glucosamine, and also those described in PCT Publication WO 02/076423 and U.S. Pat. No. 6,159,485, issued to Yu, et al. In one embodiment, the composition comprises from about 0.01% to about 15%, alternatively from about 0.1% to about 10%, and alternatively from about 0.5% to about 5%, of the sugar amine.

### Oil Control Agents

The compositions of the present invention may comprise one or more compounds useful for regulating the production of skin oil, or sebum, and for improving the appearance of oily skin. Examples of suitable oil control agents include salicylic acid, dehydroacetic acid, benzoyl peroxide, vitamin B3 compounds (for example, niacinamide or tocopheryl nicotinate), their isomers, esters, salts and derivatives, and mixtures thereof. The compositions may comprise from about 0.0001% to about 15%, alternatively from about 0.01% to about 10%, alternatively from about 0.1% to about 5%, and alternatively from about 0.2% to about 2%, of an oil control agent.

### Other Skin Care Actives

The compositions of the present invention further may comprise non-vitamin antioxidants and radical scavengers, hair growth regulators, flavonoids, minerals, preservatives, phytosterols and/or plant hormones, protease inhibitors, tyrosinase inhibitors, anti-inflammatory agents and N-acyl amino acid compounds.

Suitable non-vitamin antioxidants and radical scavengers include, but are not limited to, BHT (butylated hydroxy toluene), L-ergothioneine (available as THIOTANE™); tetrahydrocurcumin, cetyl pyridinium chloride, carnosine, diethylhexyl syrinylidene malonate (available as OXYNEX™), hexadec-8-ene-1,16-dicarboxylic acid (octadecene dioic acid; ARLATONE Dioic DCA from Uniqema), ubiquinone (co-enzyme Q10), tea extracts including green tea extract, yeast extracts or yeast culture fluid (e.g., Pitera®), and combinations thereof.

Suitable hair growth regulators include, but are not limited to, hexamidine, butylated hydroxytoluene (BHT), hexanediol, panthenol and pantothenic acid derivates, their isomers, salts and derivatives, and mixtures thereof.

Suitable minerals include zinc, manganese, magnesium, copper, iron, selenium and other mineral supplements. “Mineral” is understood to include minerals in various oxidation states, mineral complexes, salts, derivatives, and combinations thereof.

Suitable examples of plant sterols (phytosterols) and/or plant hormones include, but are not limited to, sitosterol, stigmasterol, campesterol, brassicasterol, kinetin, zeatin, and mixtures thereof.

Suitable protease inhibitors include, but are not limited to, hexamidine, vanillin acetate, menthyl anthranilate, soybean trypsin inhibitor, Bowman-Birk inhibitor, and mixtures thereof.

Suitable tyrosinase inhibitors include, but are not limited to, sinablanca (mustard seed extract), tetrahydrocurcumin, cetyl pyridinium chloride, and mixtures thereof.

Suitable anti-inflammatory agents include, but are not limited to, glycyrrhizic acid (also known as glycyrrhizin, glycyrrhixinic acid, and glycyrrhetinic acid glycoside), glycyrrhetenic acid, other licorice extracts, and combinations thereof.

Suitable N-acyl amino acid compounds include, but are not limited to, N-acyl phenylalanine, N-acyl tyrosine, their isomers, including their D and L isomers, salts, derivatives, and mixtures thereof. An example of a suitable N-acyl amino acid is N-undecylenoyl-L-phenylalanine is commercially available under the tradename SEPIWHITE® from Seppic (France).

Other useful skin care actives include moisturizing and/or conditioning agents, such as glycerol, petrolatum, caffeine, and urea; yeast extracts (for example, Pitera™); dehydroepiandrosterone (DHEA), its analogs and derivatives; exfoliating agents, including alpha- and beta-hydroxyacids, alpha-keto acids, glycolic acid and octanoyl salicylate; antimicrobial agents; antidandruff agents such as piroctone olamine, 3,4,4′-trichlorocarbanilide (trichlosan), triclocarban and zinc pyrithione; dimethyl aminoethanol (DMAE); creatine; skin lightening agents such as kojic acid, mulberry extract, hydroquinone, arbutin, and deoxy-arbutin; (sunless) tanning agents, such as dihydroxy acetone (DHA); isomers, salts, and derivatives of any of the foregoing; and mixtures thereof.

### II. Methods

The present invention describes a method of providing a benefit to mammalian keratinous tissue, including a method of protecting the mammalian skin from the effects of ultraviolet radiation, comprising the step of topically applying to mammalian skin a personal care composition described herein. Alternatively, the method may comprise the step of applying the composition described herein to insult-affected keratinous tissue, to regulate and/or improve the condition of such tissue, and/or to provide relief from the effects of the insult.

Benefits include regulating and/or improving the condition of keratinous tissue, non-limiting examples of which include reducing the appearance of wrinkles, reducing the appearance of deep lines, reducing the appearance of fine lines, reducing the appearance of large pores, reducing the thickness of keratinous tissue, increasing the convolution of the dermal-epidermal border, increasing elasticity, reducing the appearance of cellulite, reducing the appearance of discoloration, reducing the appearance of hyperpigmentation, reducing the appearance of under-eye circles, reducing the appearance of sallowness, and combinations thereof. Alternatively, the benefit may include reducing wrinkles, reducing deep lines, reducing fine lines, reducing large pores, reducing cellulite, reducing hyperpigmentation, reducing undereye circles, reducing puffiness, and combinations thereof.

The composition may be applied by a variety of means, including by rubbing, wiping or dabbing with hands or fingers, or by means of an implement and/or delivery enhancement device. Non-limiting examples of implements include a sponge or sponge-tipped applicator, a swab (for example, a cotton-tipped swab), a pen optionally comprising a foam or sponge applicator, a brush, a wipe, and combinations thereof. Non-limiting examples of delivery enhancement devices include mechanical, electrical, ultrasonic and/or other energy devices. After application, the composition may be allowed to remain on the keratinous tissue.

The amount of the composition applied, the frequency of application and the period of use will vary widely depending upon the level of components of a given composition and the level of regulation desired. For example, from about 0.1 mg composition/cm² to about 50 mg composition/cm², and alternatively about 2 mg composition/cm² of keratinous tissue may be applied. In one embodiment, the composition is applied prior to exposure of the skin to ultraviolet radiation, and alternatively at least once daily, where “daily” and “days” mean a 24-hour period. The user may be instructed to reapply the composition after a period of time has passed, for example every hour, and alternatively when the composition has been washed or rinsed from the skin, for example after washing one's hands or after swimming. The composition further may be applied as part of a treatment regimen, for example, once daily for 30 consecutive days, alternatively for 14 consecutive days, alternatively for 7 consecutive days and alternatively for 2 consecutive days.

The method may comprise the step of inducing a temperature change in the composition and/or in the keratinous tissue either simultaneously or sequentially with the step of applying the composition. The method further may comprise additional steps which form part of a treatment or application regimen, including the steps of applying at least one additional composition, ingesting one or more dietary supplements, cleansing, etc.

### III. Kit

The present invention further provides a kit comprising at least one composition described herein. The kit may comprise an outer packaging unit, which in turn may comprise one or more inner packaging units. In one embodiment, at least a portion of all packaging is transparent or translucent, such that the composition is visible to a consumer. One non-limiting example of a suitable outer container is a box or a tray, suitable for holding a sufficient number of inner packaging units for an indicated application regimen, for example, one application per day for one month. Alternatively, the tray may contain an array of individual inner packaging units which are organized to correspond to an indicated application regimen. The kit further may comprise an implement, which may be suitable for targeted delivery of the composition to a desired area of keratinous tissue. The composition may be packaged separately from the implement, or may be contained within the implement. The kit further may comprise a plurality of components, including one or more additional compositions, one or more orally ingestible dietary supplements, an additional implement, an additional delivery enhancement device, a temperature change element, a substrate, instructions for complying with suitable application regimens, and combinations thereof.

### EXAMPLES

The following describe non-limiting examples of sunscreen compositions. All percentages indicate the weight of the component expressed as a percentage of the total weight of the composition. The prepared compositions may be stored in a suitable container, and applied for example to mammalian skin exposed or expected to be exposed to ultraviolet radiation, to insult-affected skin, and/or to skin in need of a particular benefit, including aged skin.

### Examples 1-5

Anhydrous sunscreen compositions may be prepared by conventional methods (by combining all ingredients and thoroughly mixing, as would be known to one of skill in the art) from the following components:

| | Exam- | Exam- | Exam- | Exam- | Exam- |
|---|---|---|---|---|---|
| | ple | ple | ple | ple | ple |
| | 1 (%) | 2 (%) | 3 (%) | 4 (%) | 5 (%) |
| Octisalate | 4.0 | 4.0 | 4.0 | 3.0 | 4.0 |
| Homosalate | 4.0 | 4.0 | 4.0 | 3.0 | 4.0 |
| Octocrylene | 1.5 | 1.5 | 1.5 | 1.5 | 1.5 |
| Octinoxate | — | — | — | — | — |
| Avobenzone | 2.0 | 2.0 | 2.0 | 3.0 | 2.0 |
| Z-Cote ™ HP1¹ | — | — | — | — | — |
| Isopropyl Isostearate | — | — | — | — | — |
| Isopropyl | 7.5 | 7.5 | 7.5 | 15.0 | 7.5 |
| Lauroylsarcosinate | | | | | |
| Vitamin E Acetate⁹ | 0.1 | — | 0.1 | 2.0 | — |
| Dow Corning ™ 9045² | 50.7 | 43.0 | 50.6 | 48.2 | 30 |
| KSG-310³ | 20.0 | 18.7 | 20.0 | 15.0 | 31 |
| Cyclomethicone D5 | — | 12.0 | — | — | — |
| Tospearl ™ 145A⁴ | 10.0 | — | — | — | 12.0 |
| Softouch ™ CC6004⁵ | — | 7.0 | — | 10.0 | — |
| Ropearl ™ 4000⁶ | — | — | 10.0 | — | 5.0 |
| Fluoropure ™ 100C⁷ | — | — | — | — | 3.0 |
| Red Iron Oxide | — | — | 0.1 | 0.1 | — |
| Prestige ™ Fire Red | — | 0.1 | — | — | — |
| 11S2⁸ | | | | | |
| Perfume | 0.2 | 0.2 | 0.2 | 0.2 | — |
| Total: | 100% | 100% | 100% | 100% | 100% |

¹Zinc Oxide and Triethoxycaprylylsilane from BASF ™
²Cyclomethicone and Dimethicone Crosspolymer from Dow Corning ™
³PEG-15/Lauryl Dimethicone Crosspolymer and Mineral Oil From Shin-Etsu ™
⁴Polymethylsilsesquioxane from General Electric ™
⁵Boron nitride from General Electric ™
⁶Nylon-12 from Kobo ™
⁷PTFE from Shamrock Technologies ™
⁸Mica and iron oxides from Eckart ™
⁹Additionally or alternatively, the composition may comprise one or more other skin care actives, their salts and derivatives, as disclosed herein, in amounts also disclosed herein as would be deemed suitable by one of skill in the art.

### Examples 6-10

Sunscreen compositions may be prepared by conventional methods (by combining all ingredients and thoroughly mixing, as would be known to one of skill in the art) from the following components:

| | Exam- | Exam- | Exam- | Exam- | |
|---|---|---|---|---|---|
| | ple | ple | ple | ple | Example |
| | 6 (%) | 7 (%) | 8 (%) | 9 (%) | 10 (%) |
| Octisalate | 4.0 | 4.0 | 5.0 | 2.5 | — |
| Homosalate | 4.0 | 4.0 | — | — | — |
| Octocrylene | 1.0 | 1.0 | 4.0 | — | — |
| Octinoxate | — | — | — | 7.5 | 4.0 |
| Avobenzone | 1.0 | 2.0 | 1.0 | — | — |
| Z-Cote HP1¹ | — | — | — | 2.0 | 4.0 |
| Isoproyl Isostearate | — | 4.0 | — | — | — |
| Isopropyl | — | 4.0 | 10.0 | 7.5 | — |
| Lauroylsarcosinate | | | | | |
| Phenylethyl benzoate | 10.0 | 4.0 | — | — | — |
| Vitamin E Acetate⁵ | — | — | 0.1 | 2.0 | — |
| Dow Corning ™ 9045 | — | 42.0 | 49.1 | 58.5 | 40.0 |
| KSG-15² | 50.0 | — | — | — | — |
| KSG-18³ | 10.0 | — | — | — | — |
| KSG-310 | — | 20.0 | 20.0 | 10 | 15.0 |
| KSG-710⁴ | 10.0 | — | — | — | — |
| Cyclomethicone D5 | — | — | — | — | 30.0 |
| Tospearl ™ 145A | — | 4.9 | — | — | 2.0 |
| Softouch ™ CC6004 | 10.0 | 7.0 | — | 5.0 | — |
| Ropearl ™ 4000 | — | — | 10.0 | 5.0 | 5.0 |
| Fluoropure ™ 100C | — | 3.0 | — | — | — |
| Red Iron Oxide | — | 0.1 | — | — | — |
| Water | — | — | 0.8 | — | — |
| Total: | 100% | 100% | 100% | 100% | 100% |

¹Zinc Oxide and Triethoxycaprylylsilane from BASF ™
²5.5% Dimethicone/Vinyl Dimethicone Crosspolymer in Cyclomethicone from Shin-Etsu ™
³15% Dimethicone/Phenyl Vinyl Dimethicone Crosspolymer in Phenyl Trimethicone from Shin-Etsu ™
⁴25% Dimethicone/Polyglycerin-3 Crosspolymer in Dimethicone from Shin-Etsu ™
⁵Additionally or alternatively, the composition may comprise one or more other skin care actives, their salts and derivatives, as disclosed herein, in amounts also disclosed herein as would be deemed suitable by one of skill in the art.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

Whereas particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A stable personal care composition comprising:
a) from about 1% to about 20% of a non-emulsifying silicone elastomer;
b) from about 0.5% to about 10% of an emulsifying silicone elastomer;
c) from about 5% to about 30% of an oil-soluble sunscreen;
d) from about 1% to about 20% of a sunscreen solvent; and
e) from about 30% to about 80% of a non-polar emollient.

2. The composition of claim 1 wherein the composition is substantially anhydrous.

3. The composition of claim 1, wherein the oil-soluble sunscreen is selected from the group consisting of benzophenone-3, bis-ethylhexyloxyphenol methoxyphenyl triazine, butyl methoxydibenzoyl-methane, diethylamino hydroxy-benzoyl hexyl benzoate, drometrizole trisiloxane, ethylhexyl methoxy-cinnamate, ethylhexyl salicylate, ethylhexyl triazone, octocrylene, homosalate, polysilicone-15, oxybenzone, derivatives of any of the foregoing, and mixtures thereof.

4. The composition of claim 1, wherein the oil-soluble sunscreen comprises at least 2%, by weight of the composition, of a crystalline sunscreen.

5. The composition of claim 4, wherein the crystalline sunscreen is butyl methoxydibenzoyl-methane.

6. The composition of claim 1, wherein the sunscreen solvent is selected from the group consisting of butyl and isopropyl phthalimide, phenylethyl benzoate, dicaprylyl carbonate, isopropyl lauroylsarcosinate, butyl octylsalicylate, dioctyl malate, dicaprylyl maleate, derivatives of any of the foregoing, and mixtures thereof.

7. The composition of claim 1, further comprising from about 0.001% to about 40% of a particulate material.

8. The composition of claim 1, further comprising from about 0.001% to about 10% of an inorganic sunscreen, an oil-insoluble sunscreen, and mixtures thereof.

9. The composition of claim 1, further comprising at least one additional skin care active.

10. A method of providing a benefit to mammalian keratinous tissue, comprising the step of topically applying to mammalian keratinous a stable personal care composition comprising:
a) from about 1% to about 20% of a non-emulsifying silicone elastomer;
b) from about 0.5% to about 10% of an emulsifying silicone elastomer;
c) from about 5% to about 30% of an oil-soluble sunscreen;
d) from about 1% to about 20% of a sunscreen solvent; and
e) from about 30% to about 80% of a non-polar emollient.

11. The method of claim 10, wherein the benefit is protecting the mammalian keratinous tissue from the effects of ultraviolet radiation.

12. The method of claim 10, wherein the mammalian keratinous tissue is human skin exhibiting signs of skin aging.

13. The method of claim 10, wherein the mammalian keratinous tissue is insult-affected mammalian keratinous tissue.

14. The method of claim 13, wherein the mammalian keratinous tissue is human skin.

15. The method of claim 14, wherein the human skin is insult-affected human skin selected from the group consisting of sunburn, rash, shaving rash, allergen-induced rash, discoloration, dry skin, chapped skin, rough skin, and combinations thereof.

16. A kit comprising:
a) a composition comprising:
i. from about 5% to about 25% of a non-emulsifying silicone elastomer;
ii. from about 10% to about 25% of an emulsifying silicone elastomer;
iii. from about 5% to about 30% of an oil-soluble sunscreen;
iv. from about 30% to about 80% of a non-polar emollient;
v. from about 0.001% to about 5% water;
b) at least one additional component select from the group consisting of at least one additional composition, at least one orally ingestible dietary supplement, an implement, a delivery enhancement device, a temperature change element, instructions for complying with suitable application regimens, and combinations thereof; and
c) instructions for complying with a regimen to provide a benefit to keratinous tissue.
